(19) United States
(12) Patent Application Publication
(10) Pub. No.: US 2026/0060799 A1
(43) Pub. Date: Mar. 5, 2026

(54) INTRAOCULAR LENSES HAVING REDUCED OFF-AXIS PHOTIC DISTURBANCES

(71) Applicant: ForSight Vision6, Inc., South San Francisco, CA (US)

(72) Inventors: Nicole Kahn-Dror, South San Francisco, CA (US); Guy Oren, South San Francisco, CA (US); Roman Kolevatov, South San Francisco, CA (US)

(21) Appl. No.: 19/106,139

(22) PCT Filed: Aug. 24, 2023
(86) PCT No.: PCT/US2023/031047

§ 371 Date: Feb. 24, 2025

Related U.S. Application Data
(60) Provisional application No. 63/401,070, filed on Aug. 25, 2022.

(51) Int. Cl. A61F 2/16

(52) U.S. Cl. CPC A61F 2/1613; A61F 2002/169; A61F 2002/1696

(57) ABSTRACT

An intraocular lens having a solid optical component having a peripheral edge located outside of and surrounding an optical zone of the intraocular lens and an internal chamber defined by one or more interior walls located within the optical zone of the intraocular lens; and a liquid optical material contained within the internal chamber. The one or more interior walls located within the optical zone comprises a textured surface configured to diffuse light. Related apparatus, methods, and devices are provided.

### CROSS-REFERENCE TO RELATED APPLICATION

This application claims the benefit of priority under 35 U.S. C. § 119(e) to Provisional Patent Application Ser. No. 63/401,070, filed Aug. 25, 2022. The disclosure of the provisional application is incorporated by reference in its entirety.

### BACKGROUND

IOLs are typically implanted after cataract extractions. Generally, IOLs are made of a foldable material, such as silicone or acrylics, for minimizing the incision size and improving patient recovery time. Most commonly used IOLs are single-element lenses that provide a single focal distance for distance vision. Accommodating intraocular lenses (AIOLs) have also been developed to provide adjustable focal distances (or accommodations) that rely on the natural focusing ability of the eye, for example, as described in U.S. Pat. Nos. 8,414,646; 8,167,941; 9,913,712; 10,258,805; and US 2019/0269500, which are each incorporated by reference herein in their entireties.

IOLs are beneficial for patients not suffering from cataracts, but who wish to reduce their dependency on glasses and contacts to correct their myopia, hyperopia and presbyopia. Intraocular lenses used to correct large errors in myopic, hyperopic, and astigmatic eye are called “phakic intraocular lenses” and are implanted without removing the crystalline lens. In some cases, aphakic IOLs (not phakic IOLs) are implanted via lens extraction and replacement surgery even if no cataract exists. During this surgery, the crystalline lens is extracted and an IOL replaces it in a process that is very similar to cataract surgery. Refractive lens exchange, like cataract surgery, involves lens replacement, requires making a small incision in the eye for lens insertion, use of local anesthesia and lasts approximately 30 minutes.

IOLs, particularly AIOLs, may incorporate liquids in fluid chambers such that accommodation is achieved with the help of fluid-actuated mechanisms. A force exerted on a portion of the lens is transmitted via the fluid to deform a flexible layer of the lens resulting in accommodative shape change of the IOL. For example, ciliary muscle movements of the eye may be harnessed by components of an AIOL to drive shape change and accommodation. The AIOLs can achieve an optical power or diopter (D) in a desired range due to shape change of the optic upon application of a small amount of force (e.g., as little as 0.1-1.0 grams force (gf)) applied by the eye tissue. The AIOLs provide reliable dioptric change by harnessing small forces. A chamber for containing liquid materials that is formed by flexible layers of elastomeric material can change shape and thus, power of the lens depending on the volume of liquid. As fill volume increases beyond the chamber volume, the flexible layers can bulge outward creating a lens with a greater focal length.

There is need in the art for improved manufacturing of lenses that provide improved properties for patients in need. The disclosure is directed to this, as well as other, important ends.

### SUMMARY

In an aspect, described is an intraocular lens having an optical zone, the intraocular lens including a solid optical component having a peripheral edge located outside of and surrounding the optical zone of the intraocular lens and an internal chamber defined by one or more interior walls located within the optical zone of the intraocular lens; and a liquid optical material contained within the internal chamber. The one or more interior walls located within the optical zone include a textured surface configured to diffuse light.

The one or more interior walls located within the optical zone can be vertical or near vertical. The one or more interior walls can form a boundary between a first lens component composed of a material having a refractive index and a second lens component composed of a material having a refractive index that is different from the refractive index of the first lens component. The textured surface can include an irregular surface morphology. The irregular surface morphology can have an average roughness depth (R_{z}) of about 7-15 μm, an arithmetic mean roughness (R_{A}) of about 1.2-1.9 μm, maximum roughness depth (Rₘₐₓ) of about 7-30 μm, and peak density and overlap (RPC) of about 20-40 per mm.

The textured surface can be an internal-facing surface or an external-facing surface of the one or more interior walls. One or more regions of the solid optical component located outside of the optical zone can be light-diffusing via particles such as filler or pigment to scatter and/or block light entering the intraocular lens from a periphery. The one or more regions that are light-diffusing can be translucent or opaque. The one or more regions that are light-diffusing can block about 70%-100% of light rays passing through the one or more regions. The one or more regions can incorporate a pigment additive concentration that is about 0.25%-4% by weight. The lens can further include second textured surface of a wall of the intraocular lens located outside the optical zone.

In an interrelated aspect, provided is an apparatus for manufacturing an intraocular lens, the apparatus including a mold component having a cavity formed, in part, by a substantially horizontal surface and a substantially vertical surface. The substantially vertical surface is arranged to form a corresponding internal surface located within an optical zone of an intraocular lens component. At least a portion of the substantially vertical surface of the cavity includes a texture configured to imprint a corresponding texture to the corresponding internal surface that is configured to diffuse light through the intraocular lens component.

The texture can include an irregular surface morphology. The irregular surface morphology can have an average roughness depth (R_{z}) of about 7-15 μm, an arithmetic mean roughness (R_{A}) of about 1.2-1.9 μm, maximum roughness depth (Rₘₐₓ) of about 7-30 μm, and peak density and overlap (RPC) of about 20-40 per mm. The texture on the substantially vertical surface can extend less than an entirety of the substantially vertical surface forming an untextured border region on the substantially vertical surface. The untextured border region can extend between the substantially vertical surface and an adjacent second surface of the mold component. A width of the untextured border region can be about 1-50 μm. The texture configured to imprint the corresponding texture can be located directly on the at least a portion of the substantially vertical surface of the cavity. The texture configured to imprint the corresponding texture can be applied to a coating on the at least a portion of the substantially vertical surface of the cavity. The coating can include a material configured to improve release of the intraocular lens component from the mold component. The coating can include a material configured to be diamond tuned. The material can be nickel.

Transmittance of light through the corresponding texture on the intraocular lens component can be at least about 70% at 550 nm wavelength. The mold component can further include surface texture on a wall of the mold component arranged to form a corresponding internal surface located outside the optical zone of the intraocular lens component. The mold component can include a plurality of different textures applied to different surfaces. The mold component can be made of stainless steel or aluminum.

In an interrelated aspect, provided is a method of manufacturing an intraocular lens using an apparatus including a mold component having a cavity formed, in part, by a substantially horizontal surface and a substantially vertical surface. The substantially vertical surface is arranged to form a corresponding internal surface located within an optical zone of an intraocular lens component. At least a portion of the substantially vertical surface of the cavity includes a texture configured to imprint a corresponding texture to the corresponding internal surface that is configured to diffuse light through the intraocular lens component. In an interrelated aspect, provided is an intraocular lens formed by the method of manufacturing.

### BRIEF DESCRIPTION OF THE DRAWINGS

These and other aspects will now be described in detail with reference to the following drawings. Generally, the figures are exemplary and are not to scale in absolute terms or comparatively but are intended to be illustrative. Relative placement of features and elements is modified for the purpose of illustrative clarity.

FIG. 1A is a perspective view of an intraocular lens;

FIG. 1B is a side view of the intraocular lens of FIG. 1A;

FIG. 1C is a cross-sectional view of the lens of FIG. 1B taken along line A-A;

FIGS. 2A-2B show embodiments of a lens having opacified components;

FIG. 3A is a schematic of a mold for forming a solid component of a lens;

FIG. 3B is a detailed view of the mold in FIG. 3A taken at box B;

FIGS. 3C-3D are cross-sectional views of an intraocular lens having textured external surfaces near the non-optical periphery;

FIGS. 3E-3F are cross-sectional view of an intraocular lens having textured external surfaces near the non-optical periphery;

FIGS. 3G-3H are cross-sectional views of an intraocular lens having textured surfaces near the non-optical periphery between inner-facing components;

FIG. 4A illustrates a wall of a lens without any laser texture;

FIG. 4B illustrates a wall of a lens with laser texture;

FIG. 5 are light scattering intensity plots illustrating the impact of surface treatments of different types of lenses;

FIG. 6 shows the light scattering intensity of a control lens (dashed line), a laser textured lens (dotted line), and a monofocal lens (solid line) of FIG. 5;

FIGS. 7A-7E are microscopy images of representative mold texture types including a combination of dimples and domes forming rough structures, a combination of dimples and domes forming smooth structures, dimples, random pillar structures with lower peak density (RPC) and random pillar structures with higher peak density (RPC), respectfully.

### DETAILED DESCRIPTION

Intraocular lenses can cause photic phenomena such as glare dysphotopsia, straylight, surface reflections, and others that may have detrimental effect on retinal image quality. Light scattering through a lens can lead to problems of straylight within the lens. Light passing through the lens from the non-optic periphery can lead to edge-glare. A new generation of “shape change” IOLs are constructed from sealed fluid chambers within the optical region of the lens that are capable of driving changes in optical power in the eye following implantation. These fluid chambers generate refractive interfaces inside the lens within the optical region wherever the fluid and elastomer of the lens are in contact. The interfaces can also cause visual quality issues for patients, especially in the case of light entering the eye from an angle. Vertical walls formed by the solid optical components of the lens forming the internal fluid chamber within the optical portion of the lens can cause refraction of light causing halos or glare.

Straylight, glare, and halos are generally referred to in the art as positive dysphotopsias.

The intraocular lens can have an optical zone with a solid optical component and a liquid optical component contained within an internal chamber defined by one or more interior walls located within the optical zone of the lens. The solid optical component can have a peripheral edge located outside of and surrounding the optical zone of the lens. One or more interior walls located within the optical zone can have a textured surface configured to diffuse light. The diffusion of light caused by the irregular surface morphology of the textured surface(s) reduces off-axis photic disturbances, which is described in more detail herein. The one or more interior walls that are textured and located within the optical zone can be vertical or near vertical and can form a boundary between lens components. For example, the boundary can be between a first lens component composed of a material having a first refractive index and a second lens component composed of a material having a different refractive index from the first. The lens can additionally incorporate textured surfaces outside the optical zone. The textured surface can be an internal-facing surface or an external-facing surface of the one or more interior walls.

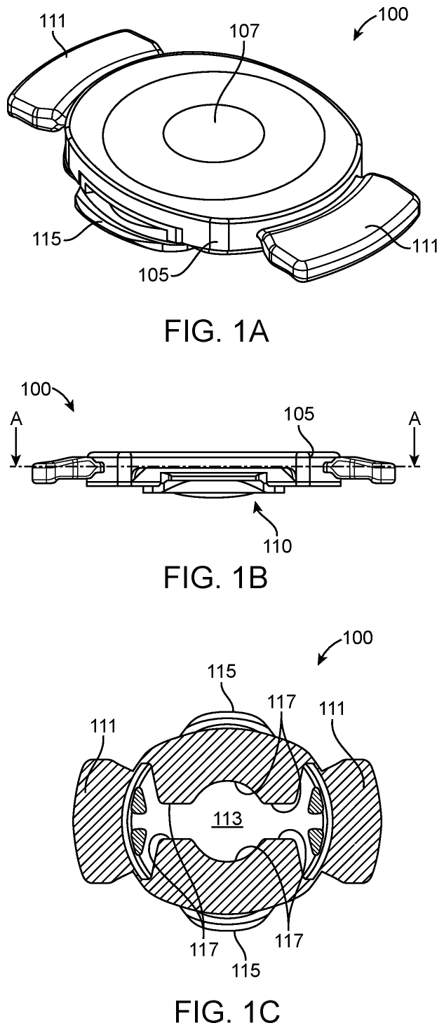

FIGS. 1A-1C illustrate an implementation of an accommodating intraocular lens 100 having internal features that refract light passing through the lens. The lens 100 can include an anterior lens capsule 105, a posterior lens structure 110, and a haptic 115. FIG. 1C shows a cross-sectional view of the lens 100 revealing an internal chamber 113 between the anterior lens capsule 105 and posterior lens structure 110 that is configured to hold a volume of liquid such as silicone oil (e.g., silicone or fluorosilicone oil). The anterior lens capsule 105 can include a dynamic anterior optic 107 and a pair of force transfer arms 111 configured to drive shape change of the anterior optic 107. The lens 100 can be formed of a material configured for small incision implantation. The solid optical components of the lens are substantially elastomeric and can be made of soft silicone polymers that are optically clear, biocompatible, and in certain circumstances flexible having a sufficiently low Young's modulus to allow for the lens body to change its degree of curvature during accommodation. Suitable materials for the solid optical component of the lens can include, but are not limited to silicone (e.g., alkyl siloxanes, phenyl siloxanes, fluorinated siloxanes, combinations/copolymers thereof), acrylic (e.g., alkyl acrylates, fluoroacrylates, phenyl acrylate, combinations/copolymers thereof), urethanes, elastomers, plastics, combinations thereof, etc. In aspects, the solid optical component of the lens is formed of a silicone elastomer, as described herein. The solid optical component can be formed of one or a combination of the materials described herein in which the liquid optical material described herein is fully encapsulated by the solid optical component. The solid optical component of a lens may include one or more regions that are configured to be in contact with and/or contain the liquid optical material. The liquid optical materials described herein can be specially formulated relative to the material of the solid optical component to mitigate lens instability and optimize optical quality. The liquid optical materials, sometimes referred to herein as an optical fluid, can include any of a variety of copolymers, including fluorosilicone copolymers and other liquid optical materials as described in PCT Application No. PCT/US2021/37354, filed Jun. 15, 2021, which is incorporated by reference herein in its entirety.

The force transfer arms 111 can have a high elongation and high tear force and can be between 30-60 Shore A durometer (e.g., MED-6233, MED-4244, MED 5/4830, MED 5/4840, MED 5/4850) in order to achieve material displacement function. The force transfer arms 111 are at the perimeter of the lens and outside the visual zone and need not be optically clear. The haptic 115, like the force transfer arms 111, need not be optically clear and is preferably opaque or translucent with a white colored pigment. The haptic 115 can be between 50-80 Shore A durometer (e.g., MED-5/4880, MED-5/4870, MED-5/4860, MED-5/4850). The dynamic anterior optic 107, in contrast, must be optically clear. The posterior lens structure 110 must also be optically clear and preferably has a high refractive index (>1.43). The posterior lens structure 110 can be between 30-70, preferably about 30-50 Shore A durometer (e.g., MED-6820, MED1-6755) whereas the anterior optic 107 can be between 30-50 Shore A durometer (e.g., MED1-6755, MED-6233, MED-6820).

The lenses described herein are designed and manufactured to mitigate positive dysphotopsias through one or more of laser texturing, light blocking masks, pigments, or dyes, and/or refractive index matching of materials. The lens 100 is described to give context to the manufacturing procedures provided herein and are not intended to be limiting. Other silicone lens types are considered that have other structural features and achieve accommodation according to other mechanisms and may be produced according to the methods described herein.

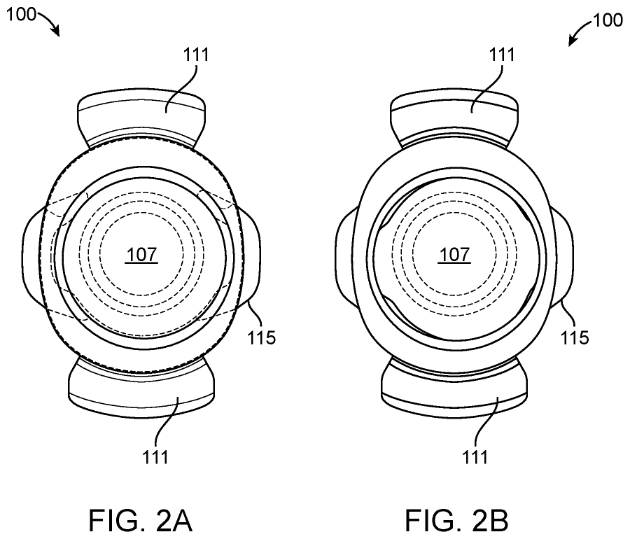

Non-optical peripheral portions of the lens such as the force transfer arms 111 or a perimeter ring of the lens such as the haptic 115 may be light-diffusing via particles such as filler or pigment or frosted to make them translucent or fully opaque. This can be achieved using a filler material, such as PDMS material with high filler content, coloring with pigment, or surface texturing. The treated portions scatter and/or block light entering the lens from the periphery. FIGS. 2A-2B are top plan views of two intraocular lenses 100. The lens 100 can include an opacified haptic 115 and transparent force transfer arms 110 and anterior optic 107 (FIG. 2A). The lens 100 can include an opacified haptic 115 and force transfer arms 110 and a transparent anterior optic 107 (FIG. 2B). The light blocking haptic 115 and force transfer arms 110 reduce edge-glare and preventing light passing through the lens from the non-optic periphery. The treated non-optic periphery can block between 70%-100% of the light rays passing through, preferably about 100%. Opacity of the treated surfaces can be adjusted for optimal performance. Light blocking effectiveness can be affected by bulk scattering characteristics, IOL geometry, and IOL position within the eye. Bulk scattering characteristics can be controlled by varying pigment and/or filler concentration, chemistry, and/or particle size. For example, PDMS can be opaque or translucent due to the usage of silica particles as a filler for improved mechanical properties. Light passing through this opaque or translucent zone is scattered thereby reducing the intensity of edge glare. The opacity can be increased further by adding a pigment additive. Pigment additive concentration can be about 0.25%-4% by weight. One or more regions of the solid optical component located outside the optical zone can also be light-diffusing via particles such as filler or pigment to scatter and/or block light entering the IOL from a periphery. The one or more regions that are pigmented can be translucent or opaque. The one or more regions can be pigmented to block about 70%-100% of light rays passing through the one or more regions, particularly to scatter and/or block light entering the intraocular lens from a periphery.

Index matching of the materials of the lens mitigates photic phenomena caused by light passing through the optical zones. Blocking light passing through the haptics mitigates photic phenomena caused by light passing through the non-optical periphery.

Roughening or texturing of the peripheral non-optical edge of lenses also helps to reduce some photic disturbances such as edge glare, it does not address the glare, reflections, refractions, and other dysphotopsias caused by the vertical or near vertical internal surfaces 117 inside the lens 100 within the optical zone of the lens (i.e., surfaces defining the internal chamber 113). These internal surfaces 117 inside the lens 100 can duplicate the mold surface texture during molding to reduce the photic disturbances within the lens caused by refractive interfaces of the solid and liquid components, as will be described in more detail below.

The lens can incorporate one or more solid components molded to form an internal chamber for containing the liquid components of the lens. The intraocular lens can be manufactured using an apparatus including one or more mold components. The mold component can incorporate one or more regions, such as a cavity formed, in part, by a substantially horizontal surface and a substantially vertical surface. The substantially vertical surface can be arranged to form a corresponding internal surface located within an optical zone of an intraocular lens component. At least a portion of the substantially vertical surface of the cavity can be textured or comprise a texture that is configured to imprint a corresponding texture to the corresponding internal surface of the molded lens component so as to diffuse light through the lens component, which will be described in detail herein.

As used herein, “diffuse” or “diffusion” of light refers to a macroscopic property of the light upon interacting with a medium or substrate (e.g., a lens component) whereas “scatter” or “scattering” of light refers to the physical description of the interaction between the light and the medium or substrate.

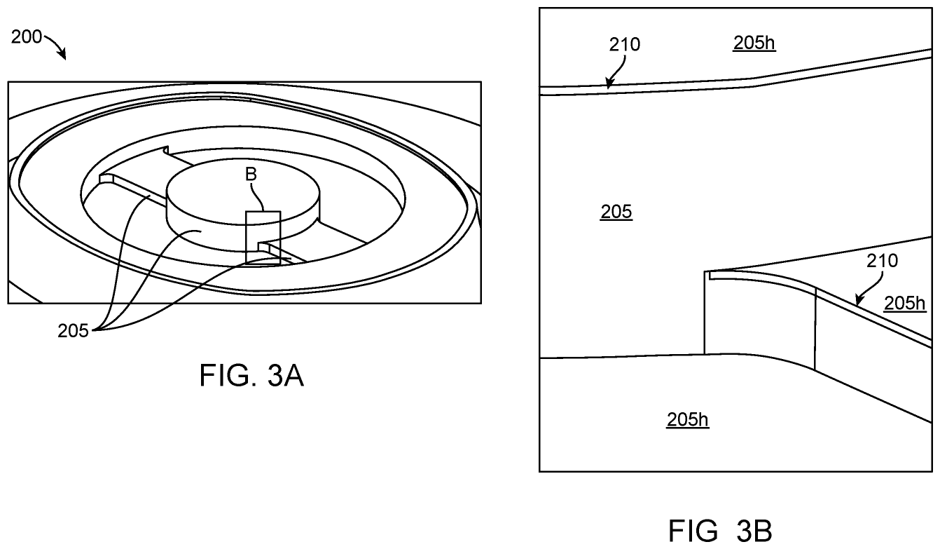

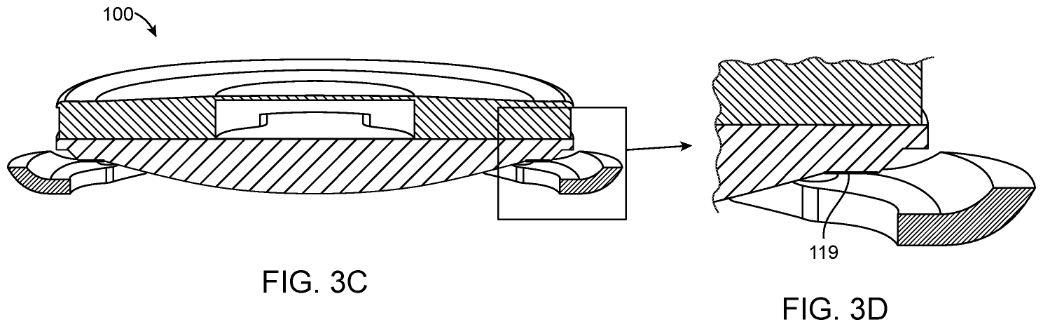

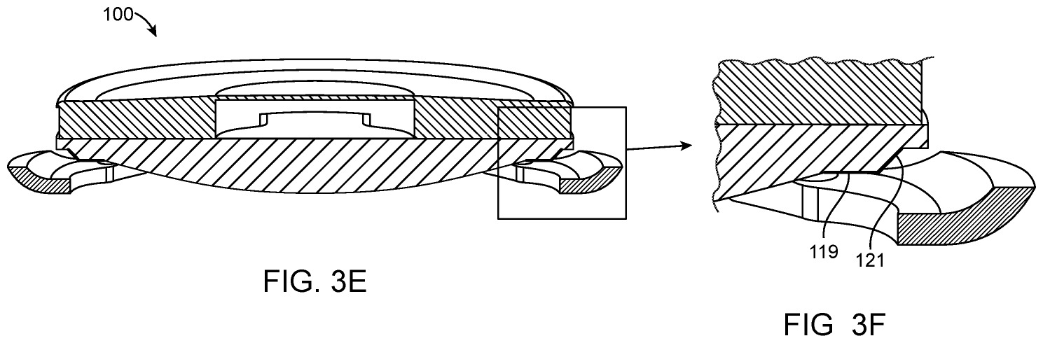

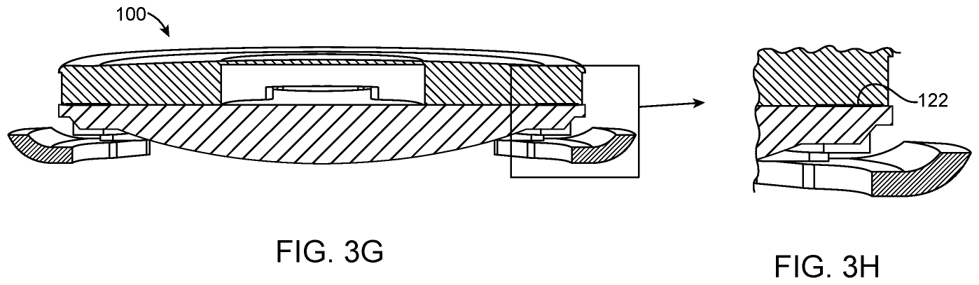

FIG. 3A is a schematic of a mold component 200 arranged to form a solid component of the lens 100. The mold component 200 is texturized along one or more surfaces 205. The texture of the mold component 200 imprints a corresponding texture or controlled microstructures onto internal surfaces 117 of the lens 100.

The surfaces 205 on the mold component 200 selected for texturing are preferably those that are arranged to form the boundaries between lens components composed of materials having different refractive indices. In particular, this is relevant for vertical or near vertical (i.e., substantially orthogonal to a plane of the anterior optic) internal surfaces 117 of the lens 100 that are within the optical zone, particularly the surfaces that define the internal chamber 113 and are thus, in contact with the silicone oil. Vertical or near vertical surfaces are preferably incorporated to form the internal chamber containing the liquid components of the lens over rounded or angled surfaces because the vertical surfaces create less glare and positive dysphotopsias in the lens. The internal surfaces 117 forming the internal chamber 113 are part of the material interface between the solid and liquid components of the lens that typically have different refractive indices. The textured internal surfaces 117 of the lens 100 diffuse light entering the lens 100 and refracting/reflecting off of the interfaces between these internal surfaces 117 and the oil within the internal chamber 113. Internal surfaces outside the optical zone may be curved and rounded or angled and slanted and may also be textured. Internal surfaces outside the optical zone can be textured as well to reduce glare from off-axis light sources.

It should be appreciated that the textured surfaces can include a wall of the lens component that is located outside the optical zone. The textured surfaces can be on internal-facing surfaces and/or external-facing surfaces of the one or more interior walls. It should also be appreciated that where a texture is described in the context of a lens component that a corresponding surface and texture is located on the mold component used in the manufacture of that lens component. Similarly, where a texture is described in the context of a mold component, a corresponding surface and texture is located on the lens component manufactured from that mold component. Thus, where the mold component is described as having a particular feature, the lens component manufactured from that mold component may similarly incorporate that particular feature even though it may not be described in the context of the lens component per se.

The mold component 200 of FIG. 3A can vary in structure depending on the final lens component shape desired. The actual structure and shape of the mold component 200 and the resulting lens 100 is not intended to be limiting and is merely illustrative to show vertical textured surfaces 205 of the mold for forming internal surfaces 117 of the lens 100 that are to be textured. Generally, the lens 100 has a solid optical component having a peripheral edge that is outside of and surrounds the optical zone of the lens. The optical zone can be defined as the diameter within a central region of the lens through which light must pass for vision and thus, must be transparent. The non-optic zone is outside the optic zone and need not be transparent.

The solid optical component includes an internal chamber 113 defined by one or more interior walls located within the optical zone of the lens. The liquid optical component of the lens is found within the internal chamber 113. The one or more interior walls located within the optical zone can include a textured internal surface 117.

A single texture can be applied to all internal surfaces of a lens, or different textures can be applied to different areas of the lens to further distribute the scattered light. For example, combining two textures that scatter light to different angles can further diffuse the light refracted through those surfaces. The mold component can incorporate a plurality of different textures applied to different surfaces. FIG. 3B is a detailed view of the mold component 200 in FIG. 3A taken at box B. The mold component 200 is texturized along one or more surfaces 205 to imprint a corresponding texture or controlled microstructure onto corresponding internal surfaces 117 of the lens 100 formed by the mold component 200. The laser texture surfaces 205 can scatter light at a first angle. The texture can extend across the entirety of the surface 205 (e.g., from a first adjacent surface 205h to a second adjacent surface 205h) or can only partially cover the surface(s) 205 between the adjacent surfaces. In the implementation in which the texture only partially covers the surface, the textured surface 205 can incorporate an untextured border region 210 between the surface 205 and an adjacent surface of the mold component 200 (see FIG. 3B). Certain surfaces of the lens structure being molded can be negatively impacted by the roughness of the texture applied. Thin membranes that are configured to flex, for example, for lens accommodation may not have the same ability to flex if laser textured. Thus, the border region 210 between the textured surface 205 and these adjacent surfaces, such as a region of the mold component 200 that creates the thin flexible membrane, mitigate these effects. The width of the border region 210 can vary from about 1 μm to about 50 μm, preferably about 5-10 μm.

Texture can also be applied to features in the peripheral non-optical edge of lenses, for example, outside the central 5 mm of the IOL up to the edges of the IOL. Textures in the peripheral non-optical edges of the lens can be advantageous to scatter light passing through the IOL periphery as can occur at large field angles. FIGS. 3C-3H are cross-sectional views of a lens like that shown in FIGS. 1A-1C where surface texture is applied to the mold surfaces forming external surfaces 119 near the lens periphery that are textured. FIGS. 3C-3D show textured surfaces 119 that are external, posterior-facing surfaces near the outer periphery of the lens. FIGS. 3E-3F show textured surfaces 119 that are external, posterior-facing surfaces near the outer periphery of the lens and also textured surfaces 121 of the lens that are facing in a more radially outward direction. Texture applied to these peripheral non-optical edges of the lens 100 aids in scattering light passing through the periphery that can occur at large field angles. FIGS. 3G-3H show texture applied to one or more internal surfaces 122 forming the interface between transparent or translucent components having different refractive indices. The interface can include that between oil and elastomer, or between two elastomeric components, such as shown in FIG. 3H.

The mold components 200 can be made of stainless steel, aluminum, plastic, 3D printed polymer, Epoxy or urethane resin, or other material or composite materials suitable for molding such materials. The mold component 200 itself may be molded and then the mold component used to mold lens components. One or more selected regions may be textured. The texture can be located directly on the mold component 200 or can be formed on a coating on the surface of the mold component 200. The mold component 200 can be textured and then coated with a thin layer of polymeric or metallic coating to improve release of the molded component from the mold component 200. Alternatively, the mold component can be textured after the application of a secondary surface coating. For example, the texture can be applied to a coating that improves the ability of the mold material to be diamond turned (such as nickel coating on stainless steel). The regions of the mold component 200 can be textured by a laser, such as an ultrashort-pulsed laser such as a nanosecond or femtosecond laser mounted on a 5-axis set-up. The laser is configured to create controlled microstructures on the mold component 200 without any heat affected zone. Alternately, the heat affected zone can be increased in a controlled way to further functionalize the surface. The texturing provided by the mold component 200 reduces unwanted photic effects without negatively impacting the release of the component part from the mold component 200 during processing. Thus, the pattern of the texture is designed to sufficiently diffuse light and allow for easy release of the part from the mold component 200. If the texture is too rough, the molded component may be difficult to release from the mold component 200. If the texture is not rough enough, the light may not be sufficiently diffused.

The scattering ability of a textured elastomeric surface is dependent on the interface between two molded lens components including surface morphology of the interface and the difference in refractive index between the two lens components. The types of surface morphologies that can be used to scatter light include irregular surface morphology to diffuse the light and regular, diffractive morphologies to distribute light between orders. The irregular surface morphology of the textured surface(s) to diffuse light can have an average roughness depth R_{z} that is between 1.5-25 μm, or about 1.0-20 μm, preferably about 7-15 μm. The regular, diffractive morphologies to distribute light between orders can have an average depth R_{z} that is between 0.5-5.0 μm. The two methods can potentially be combined to further diffuse light. The texture can be assessed using contact (e.g., Scanning Probe Microscopy) or non-contact (e.g., confocal microscopy) for various roughness parameters. The following parameters can be used to describe the surface morphology of the textured molded surfaces, including average roughness depth (R_{Z}), arithmetic mean roughness (R_{A}), maximum roughness depth (Rₘₐₓ), and peak density and overlap (RPC), as well as peak geometry, smooth or hierarchical/rough structures, and regular or random structures. The texture of the surfaces can have an average roughness depth R_{Z} that is about 1.5-25 μm or about 1.0-20 μm, or about 7-15 μm, an arithmetic mean roughness (R_{A}) that is about 0.2-4.0 μm or about 1.2-1.9 μm, and a maximum roughness depth (Rₘₐₓ) that is about 2.0-40 μm, or about 7-30 μm. The texture of the surfaces can have a peak density and overlap (RPC) that is about 16-100 per mm, or about 150-750 per mm, or more preferably about 20-40 per mm.

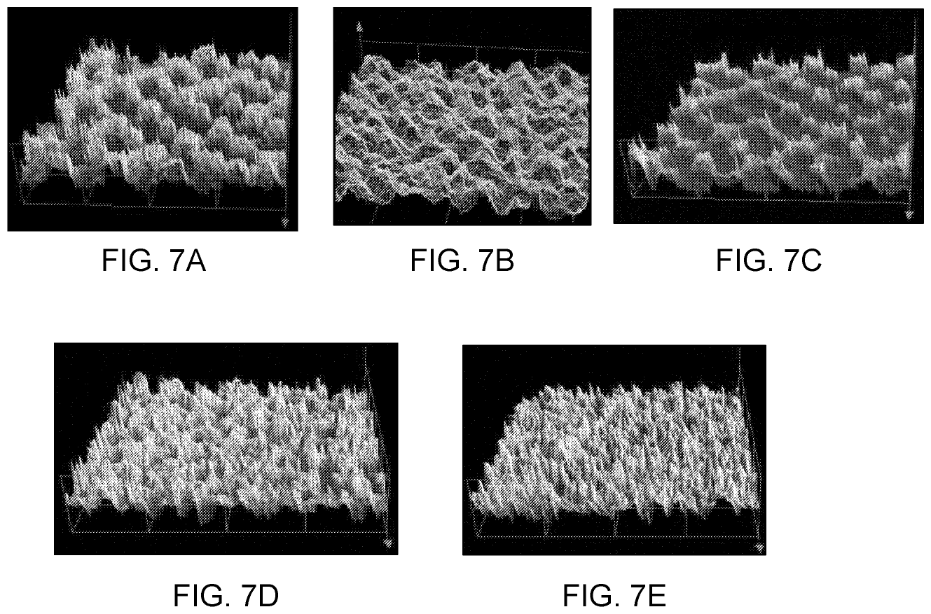

The peak geometry can include pillars, domes, and/or dimples. FIGS. 7A-7E are microscopy images of representative mold texture types considered herein. FIG. 7A illustrates a combination of dimples and domes forming rough structures. FIG. 7B illustrates a combination of dimples and domes forming smooth structures. FIG. 7C illustrates dimples. FIGS. 7D and 7E illustrate random pillar structures with lower peak density (RPC) and higher peak density (RPC), respectfully.

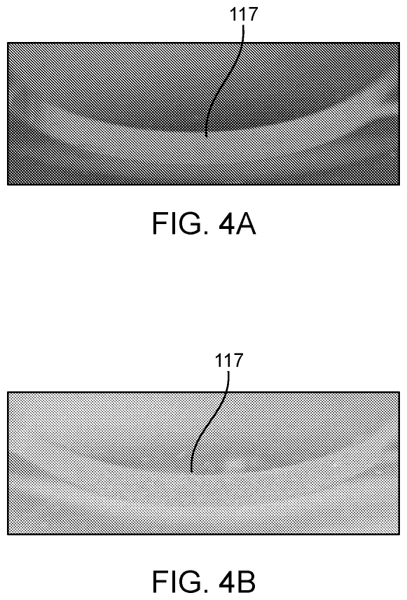

FIG. 4A illustrates a vertical wall internal surface 117 of a lens 100 without any laser texture and FIG. 4B illustrates a vertical wall internal surface 117 of a lens 100 with laser texture. Because the internal surfaces 117 are within the optical portion of the lens, the light passing through those internal surfaces 117 must be fully diffused, but without blocking the light. The transmittance of light through the internal surfaces 117 is preferably at least about 70% (at 550 nm wavelength) to prevent negative dysphotopsia.

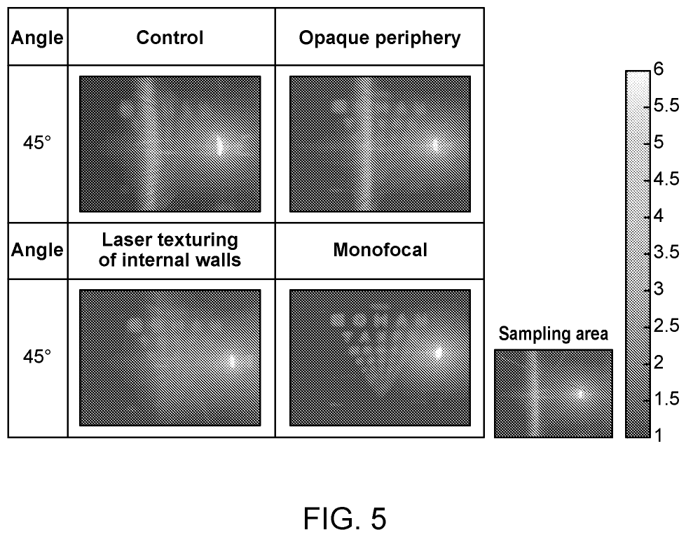

FIG. 5 illustrates the impact of laser texturing of the internal surfaces 117 of a lens in the presence of an off-axis glare source. The glare source was projected at a 45 degree angle relative to the primary optical path. The intensity of light scale is shown with light colors (top of bar on right) being higher intensity light and dark colors (bottom of bar on right) being lower intensity light. The control lens (upper left) had no surface treatments of the internal chamber walls. The control lens (upper left) has a bright glare of high intensity light, a streak of lower intensity light, and the letters in the background appear to be of relatively low visual quality. The monofocal lens (lower right) has no internal chamber walls. The monofocal lens, in contrast to the control, has only the glare of higher intensity light and the visual quality of the letters appear improved over the control. Another lens (upper right) was tested that has an internal chamber without laser texturing, but incorporates an opaque periphery similar to the lens shown in FIG. 2A. This lens still has a glare of higher intensity light on the right and a streak of lower intensity light on the left although not as severe as the control and the visual quality of the letters in the background also appear to be improved over the control lens. The lens having laser texturing of internal walls (lower left) eliminated the streak on lower intensity light on the left and reduced the glare of higher intensity light compared to control. The visual quality of the letters are better than control as well.

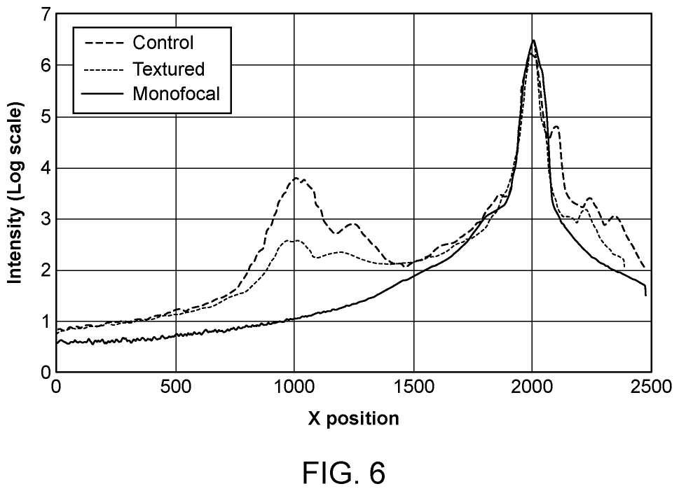

FIG. 6 shows the light scattering intensity of the control lens (dashed line), the laser textured lens (dotted line), and the monofocal lens (solid line) of FIG. 5 represented in graph form. Each of the lenses has a bright peak at about 2000× position that is substantially similar in intensity, which is the higher intensity glare shown in FIG. 5. The control and textured lenses each have a secondary peak at about 1000× position that is not present in the monofocal lens, which is the lower intensity streak shown in FIG. 5. The textured lens had a significantly reduced secondary peak compared to control. The largest peak of each of the three lenses represents the light that is transmitted through the lens without interaction with internal interfaces. The untextured lens has light interacting with the internal interfaces producing a concentrated secondary peak on the detector. The textured lens also has light interacting with the internal interface, but the texture on the walls diffused the light over a larger area of the detector. This reduced the magnitude of the secondary peak in the textured lens compared to the control lens by more than an order of magnitude. The monofocal lens does not have any internal optical interface so no second peak is visible at the selected angle.

The devices and systems described herein can incorporate any of a variety of features. Elements or features of one implementation of a device and system described herein can be incorporated alternatively or in combination with elements or features of another implementation of a device and system described herein as well as the various implants and features described in. For the sake of brevity, explicit descriptions of each of those combinations may be omitted although the various combinations are to be considered herein. Provided are some representative descriptions of how the various devices may be manufactured, however, for the sake of brevity explicit descriptions of each method with respect to each implant or system may be omitted.

In aspects, description is made with reference to the figures. However, certain aspects may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detain in order to not unnecessarily obscure the description. Reference throughout this specification to “one embodiment,” “an embodiment,” “an aspect,” “one aspect,” “one implementation, “an implementation,” or the like, means that a particular feature, structure, configuration, or characteristic described is included in at least one embodiment, aspect, or implementation. Thus, the appearance of the phrase “one embodiment,” “an embodiment,” “one aspect,” “an aspect,” “one implementation, “an implementation,” or the like, in various placed throughout this specification are not necessarily referring to the same embodiment, aspect, or implementation. Furthermore, the particular features, structures, configurations, or characteristics may be combined in any suitable manner in one or more implementations.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “front,” “side,” and “back” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples, embodiments, aspects, and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C,” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1. An intraocular lens having an optical zone, the intraocular lens comprising:
a solid optical component having a peripheral edge located outside of and surrounding the optical zone of the intraocular lens and an internal chamber defined by one or more interior walls located within the optical zone of the intraocular lens; and
a liquid optical material contained within the internal chamber,
wherein the one or more interior walls located within the optical zone comprises a textured surface configured to diffuse light.

2. The intraocular lens of claim 1, wherein the one or more interior walls located within the optical zone are vertical or near vertical.

3. The intraocular lens of claim 2, wherein the one or more interior walls form a boundary between a first lens component composed of a material having a first refractive index and a second lens component composed of a material having a second refractive index that is different from the first refractive index of the first lens component.

4. The intraocular lens of claim 1, wherein the textured surface comprises an irregular surface morphology.

5. The intraocular lens of claim 4, wherein the irregular surface morphology has an average roughness depth (R_{z}) of about 7-15 μm, an arithmetic mean roughness (R_{A}) of about 1.2-1.9 μm, maximum roughness depth (Rₘₐₓ) of about 7-30 μm, and peak density and overlap (RPC) of about 20-40 per mm.

6. The intraocular lens of claim 1, wherein the textured surface is an internal-facing surface or an external-facing surface of the one or more interior walls.

7. The intraocular lens of claim 1, wherein one or more regions of the solid optical component located outside of the optical zone are light-diffusing via particles such as filler or pigment to scatter and/or block light entering the intraocular lens from a periphery.

8. The intraocular lens of claim 7, wherein the one or more regions that are light-diffusing are translucent or opaque.

9. The intraocular lens of claim 7, wherein the one or more regions that are light-diffusing block about 70%-100% of light rays passing through the one or more regions.

10. The intraocular lens of claim 7, wherein the one or more regions incorporate a pigment additive concentration that is about 0.25%-4% by weight.

11. The intraocular lens of claim 1, further comprising a second textured surface of a wall of the intraocular lens located outside the optical zone.

12. An apparatus for manufacturing an intraocular lens, the apparatus comprising:
a mold component comprising a cavity formed, in part, by a substantially horizontal surface and a substantially vertical surface,
wherein the substantially vertical surface is arranged to form a corresponding internal surface located within an optical zone of an intraocular lens component, and
wherein at least a portion of the substantially vertical surface of the cavity comprises a texture configured to imprint a corresponding texture to the corresponding internal surface that is configured to diffuse light through the intraocular lens component.

13. The apparatus of claim 12, wherein the texture comprises an irregular surface morphology.

14. The apparatus of claim 13, wherein the irregular surface morphology has an average roughness depth (R_{z}) of about 7-15 μm, an arithmetic mean roughness (R_{A}) of about 1.2-1.9 μm, maximum roughness depth (Rₘₐₓ) of about 7-30 μm, and peak density and overlap (RPC) of about 20-40 per mm.

15. The apparatus of claim 12, wherein the texture on the substantially vertical surface extends less than an entirety of the substantially vertical surface forming an untextured border region on the substantially vertical surface.

16. The apparatus of claim 15, wherein the untextured border region extends between the substantially vertical surface and an adjacent second surface of the mold component.

17. The apparatus of claim 15, wherein a width of the untextured border region is about 1-50 μm.

18. The apparatus of claim 12, wherein the texture configured to imprint the corresponding texture is located directly on the at least a portion of the substantially vertical surface of the cavity.

19. The apparatus of claim 12, wherein the texture configured to imprint the corresponding texture is applied to a coating on the at least a portion of the substantially vertical surface of the cavity.

20. The apparatus of claim 19, wherein the coating comprises a material configured to improve release of the intraocular lens component from the mold component.

21.-28. (canceled)
